(19) United States
(12) Patent Application Publication
(10) Pub. No.: US 2025/0356971 A1
(43) Pub. Date: Nov. 20, 2025

(54) Video Documentation System and Medical Treatments Used with or Independent Thereof

(71) Applicant: P Tech, LLC, Manalapan, FL (US)

(72) Inventors: Peter M. Bonutti, Manalapan, FL (US); Justin E. Beyers, Effingham, IL (US); Anthony Caparso, San Francisco, CA (US)

(21) Appl. No.: 19/286,040

(22) Filed: Jul. 30, 2025

Related U.S. Application Data
(63) Continuation of application No. 17/401,898, filed on Aug. 13, 2021.
(60) Provisional application No. 63/065,333, filed on Aug. 13, 2020.

(51) Int. Cl. G16H 15/00; A61B 5/00; A61N 1/36; G06V 20/40; G16H 10/60; G16H 40/20; G16H 40/67; G16H 50/20; H04N 7/18

(52) U.S. Cl. CPC G16H 15/00; A61B 5/749; A61N 1/3605; G06V 20/40; G06V 20/44; G16H 10/60; G16H 40/20; G16H 50/20; H04N 7/183; G16H 40/67

(57) ABSTRACT

A video documentation system includes a camera configured to capture video of an event and a processor receiving and responsive to event data generated by the camera. The system includes computer-readable media storing an artificial intelligence (AI) system configured to generate a record of critical activities occurring during the event. The computer-readable media also store processor-executable instructions for receiving, by the AI system, the event data representative of the event from the at least one camera, processing the received event data with the AI system to identify the one or more critical activities, and providing the record of the one or more critical activities occurring during the event as an output of the AI system.

### CROSS REFERENCE TO RELATED APPLICATION

This application is a continuation application based on U.S. Non-Provisional application Ser. No. 17/401,898, filed Aug. 13, 2021, which claims the benefit of U.S. Provisional Patent Application No. 63/065,333, filed Aug. 13, 2020, the entire disclosure of which is incorporated herein by reference.

### FIELD OF THE DISCLOSURE

The present disclosure generally relates to a video documentation system. In another aspect, the present disclosure relates to a treatment which can be used with the video documentation system, such as treatment using an electrical stimulus implant.

### BACKGROUND OF THE DISCLOSURE

Medical records are typically based upon verbal documentation or time scripted documentation of an event. This is typically done after the event and done in a subjective nature—the individual, therapist, surgeon, or other healthcare provider would dictate or transcribe their summary of events. In this age of quality and metrics, the challenge really exists as to whether the healthcare provider who receives reimbursement based upon the medical record and their subjected dictated notes accurately transcribed them. In other words, the accuracy of a patient's medical records and medical procedures or treatments are based on recollection and/or honesty by the healthcare provider.

Moreover, the accuracy of records is important for other industries outside of healthcare.

### SUMMARY

In an aspect, a video documentation system comprises at least one camera configured to capture video of an event and to generate event data representative thereof. One or more processors coupled to the camera receive and are responsive to the event data via a communications network. The system also includes one or more non-transitory computer-readable media coupled to the processors for storing an artificial intelligence (AI) system configured to generate a record of one or more critical activities occurring during the event. The non-transitory computer-readable media also store instructions that, when executed by the processors, configure the system to perform operations. The operations comprise receiving, by the AI system, the event data representative of the event from the at least one camera, processing the received event data with the AI system to identify the one or more critical activities, and providing the record of the one or more critical activities occurring during the event as an output of the AI system.

A method embodying aspects of the present disclosure generates a record of critical activities occurring during an event. The method comprises receiving, by an artificial intelligence (AI) system, event data representative of the event. The event data is received from at least one camera configured to capture video of the event and to generate the event data representative thereof. The method also includes executing, by one or more processors, instructions stored on one or more non-transitory computer-readable media to configure the AI system to perform operations. The operations performed by the AI system comprise processing the received event data with the AI system to identify the critical activities and providing the record of the one or more critical activities occurring during the event as an output of the AI system.

Other objects and features will be in part apparent and in part pointed out hereinafter.

### BRIEF DESCRIPTION OF THE DRAWINGS

FIG. 1 is a schematic representation of a documentation system and associated systems and components in wired or wireless communication with the documentation system;

FIG. 2 is a schematic representation of components of the documentation system in the environment of an operating room;

FIG. 3 is a schematic representation of the documentation system and associated systems and components in the context of an implantable device and/or sensor;

FIG. 4 is a schematic representation of one embodiment of an implantable device;

FIG. 5 is a schematic representation of a hand-held source of power for the implantable device;

FIG. 6 is a schematic representation of another embodiment of a hand-held source of power for the implantable device;

FIG. 7 is a schematic representation of an exemplary treatment using the implantable device;

FIG. 8 is a schematic representation of implanted sensors;

FIG. 9 is a schematic representation of an indwelling vascular access catheter;

FIG. 10 is a schematic representation of the indwelling vascular access catheter placed in a patient; and

FIG. 11 depicts an audio and/or visual editing and sharing application or platform.

### DETAILED DESCRIPTION OF THE DISCLOSURE

The present disclosure is directed to a documentation system for patient medical records, insurance compliance for healthcare providers, medical diagnosis, therapy, surgery, general healthcare, teaching, and/or other purposes. In one aspect, video is selectively recorded during an “event.” As used herein, an “event” is any activity that is desired to be documented, such as a surgery, a therapy session, a teaching session, a diagnosis or diagnostic testing, etc. The video recording or data of the event, which is preferably digital but may be analog and converted to digital, is analyzed by software to provide useful, user-friendly information to a user for a specific purpose. This information may be analyzed and provided to the user intraoperatively or post-operatively. For example, and explained in more detail below, the specific purpose(s) may be patient medical records, medical quality of care, insurance compliance for healthcare providers, medical diagnosis, therapy, teaching, and/or other purposes.

### Analysis Software

The following examples relate to examples of analysis software of the video documentation system for analyzing video data. In the video documentation system of present disclosure, one or more of these examples may be incorporated and combined therein. The software may be artificial intelligence developed using machine-learning techniques, such as those described in U.S. Pat. No. 10,402,748, the entirety of which is hereby incorporated by reference. Other analysis software may be incorporated in the video documentation system. For example, Suitable AR/VR methods and systems for use with the disclosed video documentation system are disclosed in U.S. Patent Application Publication No. 2019/0065970, the entirety of which is incorporated by reference herein.

In one example, the analysis software is configured to determine critical activity or activities during the event and automatically cut the video data so that only the critical activity or activities remain in the outputted “analyzed video data” to be used by the user. The software may be AI software capable of recognizing selected critical activities during the event. In one embodiment, the video documentation system may be configured for a specific surgery. The video data may include both visual data and audio data, each of which may be analyzed to determine or find the critical activities. This information may be analyzed by the software and provided to the user intraoperatively or post-surgery.

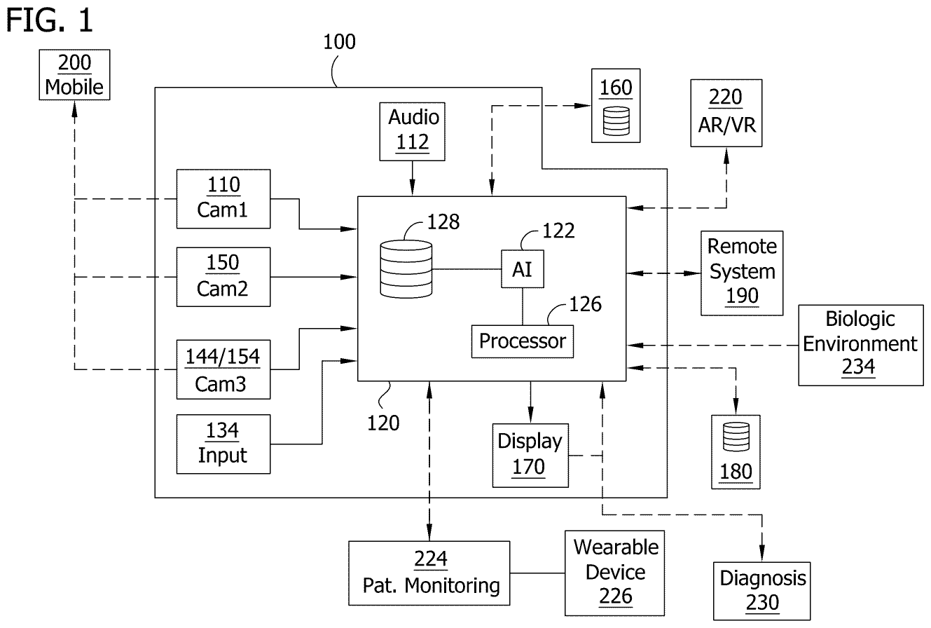

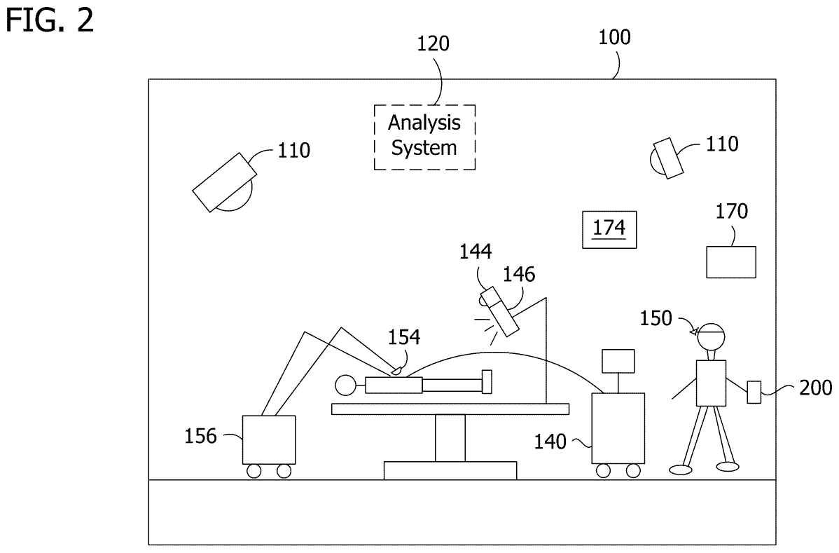

For example, the entirety of a surgery may be videoed (e.g., visual and audio data). Referring to FIGS. 1 and 2, an exemplary system documentation system is indicated at reference numeral 100. The illustrated system 100 includes, among other components, one or more cameras 110 (broadly, image sensor), an audio input 112 (which may come from the camera), and an analysis system 120. The analysis system 120 may include, among other components, the analysis software 122 (e.g., AI software), a processor 126, and a database 128. The data from the event (e.g., procedure) is saved in the database 128. This database 128 is accessible by the processor 126, which runs the analysis software 122.

The analysis software 122 analyzes the video data and recognizes selected critical aspects. The software 122 automatically bypasses or cuts out the sections of video that are not essential or reasonable or relevant to quality or treatment, and identifies the critical aspects to shorten or focus the reviewer via either computer review through artificial intelligence or manual review. This could be done through artificial intelligence by mapping of large data points to determine standards, metrics, and disease profiles. Other known AI methods could be implemented. This could be done for any type of procedure including in-office procedure, diagnostics and evaluations of patients. An alternative embodiment could put markers at key points in the video, allowed the review to skip to relevant sections automatically. Another embodiment would increase the playback speed during non-critical sections.

For example, the software 122 may recognize the “timeout,” which in general is period of time when the surgeon states the patient's name and surgery being performed, for example. The analysis software may be configured to recognize when the surgeon is talking during the timeout, and identify and record this period of time as the timeout. The analysis software 122 may use voice recognition to perform this task. In one embodiment, the patients name could be extracted from the timeout and used to query the medical records to confirm details about the procedure to be performed. It is also considered that the information from medical records, or information extracted from the timeout could be used augment program flow The software 122 may be configured to perform speech recognition to identify the timeout. In another example, the surgeon or other person may be required signal or identify the timeout for the system. This identification can be performed by voice command or manual input 134 into the system or a movement command. The analysis software 122 is configured to recognize this command or identification. The software 122 may be further configured to analyze the timeout activity to determine if it was performed correctly (e.g., determine if the surgeon performed the timeout correctly and the name and surgery to be performed matches surgery data). This information may be analyzed and provided to the user intraoperatively or post-surgery. The information recorded during this section could be compared to patient information via HL7, DICOM or other known healthcare information system (HCIS) protocols to verify patient information, and to pull in other available information about the patient and/or procedure.

In addition to or alternatively, the software 122 may be configured to recognize other critical aspects of the recorded surgery (or recognize commands given by the surgeon or other person) that it is programmed to recognize, using visual data and/or audio data. For example, a critical aspect may be visual data of the tissue to be operated on (“target tissue”) before surgery is performed on the tissue for purposes of diagnosis, for example. Thus, the software 122 may be configured to recognize the target tissue when the surgeon has visualized the target tissue before the surgery has started. This video could come from cameras 110 mounted in the room, an endoscope 140, or any camera (e.g., camera 144 mounted on a light 146; camera 150 mounted on surgeon (such as head or visor) or other healthcare practitioner; or camera 154 mounted on a surgical robot 156) used during the surgical procedure. The software 122 may be further configured to analyze the visualized target tissue to diagnose the target tissue and/or determine if a pre-operative diagnosis of the target tissue is accurate. As shown in FIG. 1, the system 100 may link to a database 160 (e.g., query a remote database) that includes the patient's diagnosis (or diagnostic data such as data from a CT, MRI, ultrasound, endoscopy, etc.) or the diagnosis may be inputted into a database 128 of the system. This automatic analysis can be used by a user to determine one or more of i) whether the pre-operative diagnosis was accurate, ii) whether an intraoperative diagnosis is accurate, and iii) whether the surgery performed (or a decision to not perform surgery) was appropriate. This information provided by the system 100 can be used by insurance companies, hospitals, teaching institutions, etc. This information may be analyzed and provided to the user intraoperatively or post-surgery. As an illustrative non-limiting example, AI software 122 is developed by analyzing numerous videos of the type of injury or other diagnosis so that the software is capable of using contemporaneous visual data being analyzed to recognize a proper diagnosis.

In another non-limiting example, a critical aspect may be visual data of the target tissue (and steps performed by the surgeon) during surgery for purposes of determining whether the procedure was adequately performed, for example. Thus, the software 122 may be configured to recognize main or pre-selected steps performed during the procedure. The software 122 may be further configured to analyze the steps to determine one or more of i) whether the steps of the procedure were performed (or are being intraoperatively performed) adequately; ii) whether required steps were performed (or are being intraoperatively performed); iii) the order of the required steps (e.g., were the steps performed in the correct order); iv) whether a procedure was actually performed. The software 122 may be configured to identify and communicate which steps were performed adequately and which steps were not or may not have been performed adequately. For example, the software may flag a step or procedure as possibly not being performed adequately. This information provided by the system can be used by insurance companies, hospitals, teaching institutions, etc. This information may be analyzed and provided to the user intraoperatively or post-surgery. As an illustrative non-limiting example, AI software 122 is developed by analyzing numerous videos of the type of surgery being performed so that the software is capable of using contemporaneous visual data being analyzed to recognize a proper surgical procedure. It is also considered that the output of the system 100 could be used to create a subversive virtual reality training tool. In another embodiment, augmented reality can be used to give the physician real time information via a display 170.

Another embodiment would use voice analysis either from the video stream or with separate microphones 174. The software 122 could monitor for changes in voice pitch and timing as an indicator of stress or abnormal behavior by the physician, patient, or support staff. This information could be used to indicate possible areas of interest on the video.

In another non-limiting example, post-operative data for purposes of determining whether the procedure was adequately successful, for example, may be inputted into the system 100. The post-operative video data may include visual and audio data, including voice recognition of the patient when describing his/her outcome, such as pain, stability, or other characteristics. The documentation system 100 may be linked to a remote database 180, for example, to query additional post-operative data (e.g., diagnostic data such an imaging data, bloodwork, etc.). (This remote database 180 may be in addition to the remote database 160 storing the pre-operative data, or the databases may be combined in a single database.) The software 122 may be further configured to analyze the post-operative video data to determine one or more of i) whether the patient has a subjectively adequate outcome; ii) whether patient has an objectively adequate outcome; iii) whether any post-operative diagnosis or complication is accurately identified. This information provided by the system 100 can be used by insurance companies, hospitals, teaching institutions, etc. As an example, the system 100 may be linked or capable of communicating with remote systems 190 at one or more of insurance companies, hospitals, teaching institutions, etc. This information may be analyzed and provided to the user intraoperatively or post-surgery. As an illustrative non-limiting example, AI software 122 is developed by analyzing numerous videos of the type of surgery being performed so that the software is capable of recognizing whether a surgical procedure has an adequate outcome.

This system could also be used to optimize efficiency and minimize complications. Procedures or visits with post-operative complication, excessive length, or low patient satisfaction would be noted in the database along with procedures with higher success rates, more efficient times, and high patient satisfaction. As a large data set is created, the information would be weighted to create an optimal procedure flow for each case. During a procedure or clinical setting, if a physician or support staff varies too far from predetermined steps in a procedure or missed a step, the system may generate a summary of possible improvements during the treatment or surgery (such as via the display 170), at the end of the treatment or surgery, and/or at the end of the day or week. If an action was performed that was too far outside the standard practice or if an action had been predictive of a critical complication, an immediate alert could be sent to a phone, smart watch, or a device (e.g., device 200) to give tactile or audible feedback during the procedure. For example, if the healthcare provider failed to request certain diagnostic testing or as certain questions during a patient visit based on the patient's verbal symptoms and/or diagnostic results, the system 100 may generate information in that regard during the visit for the provider to correct any omissions or mistakes. The system 100 could constantly update based on outcomes to ensure evolve the algorithm.

In one embodiment, as described above and shown in FIGS. 1 and 2, the software 122 may analyze pre-operative data (e.g., video data and/or other diagnostic data), intraoperative data (e.g., video data and/or other diagnostic data), and post-operative data (e.g., video data and/or other diagnostic data). Thus the software 122 may be capable of analyzing all aspects of a surgery to give an overall outcome rating or determination.

In one aspect, the video information collected by the system 100 creates a labeled data set for machine vision. Creating a large labeled dataset of images is very valuable when training a convolutional neural network for machine vision or detection. Video or visual images taken before and after surgery, such as meniscal repair for proving a correct procedure was performed, for example, can be used to create a labeled dataset. As surgeons continue to label and submit these pictures, a large data set can be created to train a convolutional neural network of the system that could be used for insurance verification or even computer navigated surgeries. This would be a similar technique to the Captcha system that was created to verify that you are a real user on a website. This system 100 was used to prevent automated robots from accessing websites, but it also created an extremely large labeled dataset of stop signs, mountains, crosswalks, etc. that were then able to be used for training self-driving cars. Having the physician label these pictures to ensure that the billing was correctly done would create a very large and accurate image and movie dataset that would allow for advancement in medical imaging, and surgical robotics.

As a non-limiting illustrative example, the surgery may be a meniscectomy. The documentation system 100 may be used to determine whether a diagnosed meniscal tear (pre-operative or intraoperative diagnosis) was consistent and whether the meniscus was removed appropriately and completely. This analysis could be done via video overlays through artificial intelligence or through knowing patient's size/weight demographics or through other analytical software and then counterchecked these so the insurance carrier or quality of care at the hospital can be evaluated. The information communicated would indicate whether there was a meniscal tear or the meniscus was not removed appropriately or there was other pathology that was missed for example. In one example, there may be a secondary individual that would over check to determine accuracy, quality, and completeness of the procedure. Billing, such as by an insurance carrier, may be appropriate or denied based on lack of or failure to perform a reasonable procedure. As can be understood, the video documentation system can be applied to any surgical procedure.

In another example, the documentation system 100 may be utilized in a clinical or office visit setting. For example, at a doctor visit, the doctor is billed for so many minutes with the patient and they have to do so many “bullet points” or evaluate diagnostic issues. Rather than the doctor dictating “I looked at the scan, blood vessel, neurologic exam, psychology exam and bill an extensive exam”, one would now have video documentation that would standardize this. Rather than relying on the doctor or healthcare practitioner to dictate or write a note, analysis of a video recording of the visit allows for objective information to be produced. For example, the audio portion of the video can be analyzed by the software, using voice recognition for example, to confirm that the practitioner adequately communicated pre-selected information to the patient. Moreover, the video portion of the video can be analyzed by the software to determine procedures performed on the patient. The practitioner may audibly discuss during the procedure and the audible segments could be focused in on a brief note and then have a video backup to determine if the healthcare provider “did what they said they did.” Backup processes, whether software based or manual based, may double check or overlie this information. With manual overview allowing the reviewers, for example it could even be a nurse that would look over this, but they would have templates to help them determine if the diagnosis was accurate and the procedure was done appropriately as well as if the rehabilitation or treatment was done appropriately.

In terms of medical diagnostics, the video documentation system may be linked with (e.g., in communication with; e.g., capable of querying) the remote database 160 including, for example, data from a CT, MRI, ultrasound, endoscopy, etc. This data may include visual and/or audio data. The software 122 may be capable of making or indicating a diagnosis. This diagnosis and/or data can be used by the video documentation system during the surgery, as outlined above.

In one example of a clinical or doctor visit situation such as when the patient returns for a visit after treatment or surgery or another situation, software 122 can compare one video of an activity to another video and/or audio of an activity and be able to search quickly so that these two sections could overlap to compare and contrast. Machine learning and artificial intelligence software is configured to extract portions of visual data and/or audio data to overlay the sets of data and determine differences between the previous visit and the current visit. This could be used in depth either through either basic stick marking figures that would give you a general overlap of the first and the second so you may not be overlapping the actual videos themselves, but recreations that would show you for example what the joints would look like with range of motion or functional activity, how the spine is flexed/extended, or what the finger/shoulder motion is. These videos could be captured simply from an IPhone or Android device or it could be a series of cameras setup in a specific array in the room that the patient would come from one visit and then come to the next visit. The patient could then input data from home off their IPhone or Android device virtually to a site where it would be analyzed and linked onto existing videos that are in the practitioner's office, insurance carrier's office, or to a cloud based system that would link the two and look for differences. This could be used for diagnostic purposes i.e. specific limping patterns that would give us specific x-rays, MRI, or CT scan or it would look also at the patient's pain trying to determine subjective and objective determinations of pain by overlapping one video versus another looking for distances, facial issues, sweating issues, thermal recognition issues, vasodilatation so we could look very close at skin for example, cilia or hand markings, or more distance views. The machine learning and artificial intelligence software is configured to determine between one view and another whether there are distance or angular changes but map them out so that these could be looked at on a true objective basis to compare one to the next to look for subtle differences and see if the patient is improving or getting worse.

The video documentation system can be used for patient records or medical documentation. Audio data and/or video data is used by the system so the physician does not have to write anything and it would actually be far more accurate recognition to what the patient did or said. For example, if one has a twenty-minute evaluation of a patient, the challenge is how you review the relevant audio and video components of that and how do you know which segments of this to store. The software is configured to recognize the critical aspects and remove the segments that are not necessary in store only integral segments of the video and/or audio so at the next visit if there are any challenges or if there are any issues one could automatically link to that specific complaint or that specific problem and then this would fast-forward to that video/audio segment to allow easy comparison of one to the next and allow us to diagnose. Therefore, rather than writing down observations which can be erroneous or inaccurate, one would have true video/audio representations so that one could be more accurate. For example, when someone says something such as my back hurts but the way they say it on how you would located it. They would say my back hurts, but when they point specifically they may point to the sacroiliac joint. Having that on a video would save, but an office note may say low back pain. It may be written in HCPCS code, but this would not be accurate. Here, it would be accurate because you actually see where the patient is pointing and what they are doing as well as how they could overlay that to the next video from the next visit and how that can be fast-forwarded so there is not a lot of time wasted. This would be a more accurate and better documentation for this.

In one example, the documentation system 100 is configured to link a specific diagnosis or procedure based on the video analysis to HCPCS codes or medical billing codes so that they would be more accurate. For example, if the patient did discuss peripheral edema or that you saw peripheral edema on the exam and it is video captured then this could be linked to severity and to HCPCS code that would be exact relative to what the patient is describing and how you are treating it. Right now, with subjective, this would be truly objective observations as well as video documentation. Again, how to narrow this streamline and also to encode it so it would not take up so much storage space. Over time, the storage space would not be required. This would be eliminated and only key features that were listed on the HCPCS code could be stored in the long-term data algorithm so one could compare one to the next based on video/audio link to some type of diagnostic code and treatment code.

The documentation system 100 can be used outside the medical space. For example, it could be done for any educational program, school systems, and special education. If someone is claiming they did a certain process and there are questions whether this was actually done or for legal situations and legal documentations, this could eliminate the need for a transcriptionist for example during subpoenas or during questions or inquiries. Policeman currently use bodycams for example to evaluate incidents and episodes. These could, however, be more routinely done but through artificial intelligence and through standardization of linking audio, video, and peripheral diagnostics or evaluation systems such as sonar radar, etc. This could be linked altogether. Artificial intelligence with standard norms could be applied to see if something falls outside the standard or something was discussed outside the standard as well as something being physically performed outside the standard. One could then assess these issues for quality metrics, value, and/or potential reimbursement.

### Camera Hardware

In one example, the one or more video cameras 110, 150, 144, 154 (i.e., imaging devices) are in communication (e.g., wired or wireless) with the analysis system 120 to store the video data in the database 128. The database 128 may be remote (e.g., cloud based) from the other components of the documentation system and in communication therefore (e.g., wired or wireless communication) or a part of the system. The camera may be digital or analog. Examples of cameras and locations thereof are detailed below, with the understanding that any combinations of cameras and other cameras are contemplated.

As an example, one or more cameras may be positioned within an operating room and may capture the surgeon and others performing the surgery, as shown in FIG. 2. This may give a broader perspective of the surgery.

As an example, one or more cameras may be positioned or positionable on the user, such as a healthcare practitioner. The camera 150 may be operatively coupled to the head of the practitioner, such as on goggles or glasses or a head band, or other locations on the practitioner. The camera may be mounted on a chassis to reduce or dampen excessive movement of the camera or the camera may include software to reduce excessive movement in the video data. In one embodiment, the camera is located to capture the point-of-view of the user. This would force the user's positions, etc. to truly visually document what they claim they are documenting.

As an example, the one or more cameras 150, 154 may be positioned on the endoscope 140 or robot 156 for assisted surgery or other instrument or device that is insertable into the patient's body to obtain video of the target tissue.

One or more of the 2 camera(s) may be 3-dimensional versus 2-dimensional cameras. Any suitable number of cameras may be used. The cameras may be fixed in multiple quadrants of the room so one could determine where the patients moves relative to fixed objects in the room, i.e. 90 degree wall, 90 degree angle, floor, ceiling, and wall so one could extrapolate actual motion patterns based on external geometry to the room.

The camera(s) can be linked to a mobile device or mobile phone 200 again storing in the cloud and being able to program these to specific files and then link those files to the next visit or the next evaluation. This could also be done for non-medical purposes such as evaluating individuals at work, work function, or work activity. This could also be done to train employees to do certain functions. It could also be linked to exoskeletal functions. One could link these to EMGs so for muscular motion patterns. If one wants to program specific motion pattern from employee on exoskeleton or motion pattern for doing some type of complex activities, one could video those motion patterns and then put wearable gloves that would give stimuli to encourage the employee to move in a certain fashion repeatedly so educate muscle groups either through stimulation motion or through confirmation of video/audio so this would again link to possible old exercise patents that we filed.

### Solutions/Benefits of Disclosed Video Documentation System

Many of the keys are to figure out how to shorten this through technology with automated review and then questions would override and then create templates of video/audio diagnostic etc. so that is something falls outside the standards it would alert the physician, surgeon, reviewer, etc. that they need to forward this. In addition, it would force the surgeons, physicians, providers, policeman, etc. to focus on these critical areas of documentation rather than simply give a secondhand dictator review which is essentially subjective interpretation of what a patient or individual says/does and is not accurate. Therefore, it is not quality based. In this era, quality based metrics have changed. Many of these artificial intelligence programs are already done in a piecemeal fashion; but, no one has coordinated all these to have the voice recognition and keywords identified and focus doing the same for diagnostic procedures for example MRI, CT, x-rays, etc. linking together and then in addition adding these to video segments and/or pictures to prove certain parts of the procedure are done appropriately. Insurance carriers can then reduce costs substantially and providers will be forced during the procedure to “prove” pathology and “prove” they did what they said they did. It would save the individuals treating the patients substantial time because they will not have to dictate a subjective note. This could all be incorporated into one formatted program that would be far more accurate and helpful. Also going downstream if pathology is missed, one could re-review these to determine if the pathology was accurate or if something else could be gleaming from the data downstream. One could create pixels of this or move these pixels.

This would really help linearly for true patient care. For example, if someone had an injury ten years ago and then could come back and look at all these parameters which are now stored in a more accurate fashion one would be able to perform a better treatment program or assess the patient's history and/or pathology based on objective data not subjective notes which are physicians interpretations. This would all be objective data. Some of these are occasionally stored such as arthroscopic videos and other clips, but linking these altogether including voice, video, and diagnostics and then using artificial intelligence to focus on certain key elements to save or store these so repeated exams would be much simpler and faster for the surgeon, physician, treating individual, legal/medical purposes, or insurance purposes. This would save substantial personnel times, especially as we go toward telemedicine and remote medical care.

This is contemporary documentation so this would be the most accurate. The contemporary documentation of both audio and video and then could be able to certify that into actual work that is done and linked this/overlay it to other diagnostic procedures, x-rays, MRI, and CT. This then would also be linked to Telemedicine, what the patients can do at home and how to rapidly search and rapidly overlay this so one would have a better idea of functional status as one would also video the exam whether it is patient's walking or general surgeon examining the abdomen, or whether a neurologist is looking at the head, neck, and face to determine whether there is any stress or psychological issues and/or to link or overlay that to diagnostics and/or prior procedures or the requirements for future procedures.

As shown in FIG. 1, the system can be used in combination with or within one or more systems. For example, the system and methods of navigation and visualization 220 set forth in U.S. Pat. No. 10,058,393, the entirety of which is incorporated by reference herein, can be modified or used in combination with the present system. In another example, the patient monitoring system 224, which may include an orthosis or other wearable device 226 (e.g., watch, heart monitor, pulse monitor, etc.), as set forth in U.S. Pat. No. 10,058,393, the entirety of which is incorporated by reference herein, can be modified or used in combination with the present system. In yet another example, the system 230 and method for use in diagnosing a medical condition of a patient, as set forth in U.S. Patent Application Publication No. 2014/0276096, the entirety of which is incorporated by reference herein, can be modified or used in combination with the present system. In yet another example, the robotic system and methods 156, as set forth in U.S. Pat. No. 9,155,544, the entirety of which is incorporated by reference herein, can be modified or used in combination with the present system. In yet another example, the methods and devices for controlling biologic microenvironments 234, as set forth in U.S. Pat. No. 8,641,660, the entirety of which is incorporated by reference herein, can be modified or used in combination with the present system. Any or all of the above can also be combined.

### Examples of Medical Device and Treatment Using Energy Impulses to Bodily Tissue

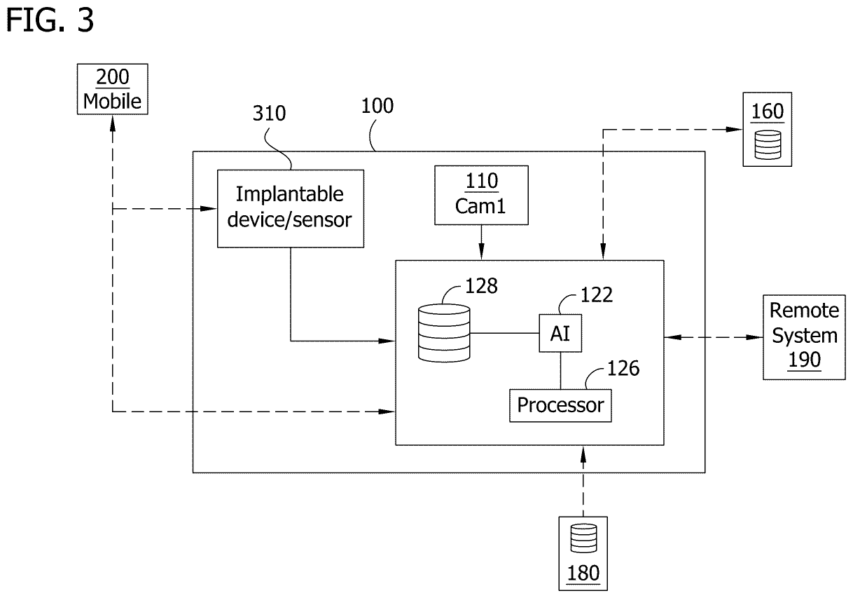

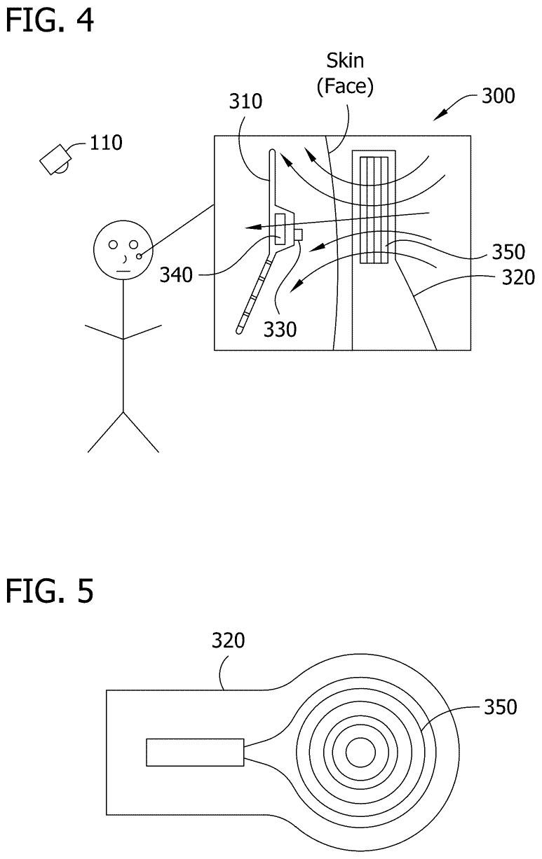

In one example, a suitable treatment for use with the video documentation system or used independent of the system relates to delivery of energy impulses (and/or energy fields) to bodily tissues for therapeutic purposes and, more particularly, to the use of electrical stimulation of the sphenopalatine ganglion (SPG) and other sensory and autonomic nerves for treating disorders in a patient and/or to increase blood flow after a stroke. A suitable device for performing such treatment is disclosed in U.S. Patent Application Publication Nos. 2019/0290908 and 2019/0201695, the entirety of each of which is incorporated by reference herein. An example of this device is indicated generally at reference numeral 300 in FIG. 4. The device 300 includes an implant 310 and a wireless source of energy 320 configured to supply energy to the implant for electrical stimulation. The implant 310 may include a sensor 330 for supplying input data to the user and/or the documentation system 100. FIG. 3 illustrates an example of the system 100 showing the implantable device 310 being part of the system and other remote components that may be in communication with the system, as described above.

Referring to FIG. 4, with respect to the treatment of a stroke, an implantable device 310 may be configured to provide parasympathetic stimulation to cause cranial blood vessel dilation without edema, thus treating vasospasm. The therapy would be a low frequency stimulation to the SPG, vidian nerve, or to the mixed nerves that exit the SPG and go into the cranium, including nasopharyngeal nerve and others. For example, periodic low frequency stimulation in the range of 1-50 Hz, and more specifically in the 5-20 Hz range would effectively cause dilation in the cerebral vessels. The therapy may be positioned ipsilateral to the side of stroke, with the understanding that the SPG innervation is not limited to the ipsilateral side only, there is some cross coverage in the innervations. Another embodiment could stimulate the stellate ganglion. Also, the stimulation can be done in concert with cardiac output, as to not cause significant hemodynamic changes to the patient, which is one reason by period stimulation is preferred over continuous stimulation as it relates to vasospasm. A camera 110 or other sensor may be used to collect data regarding the treatment and progress of the patient for use with the documentation system 100, as described above. For example, the software 122 of the documentation system 100 may analyze progress made due to the treatment and/or progress made during treatment.

In one or more embodiments, the implantable device 310 may include coils 340 or one or more flex circuits, rather than copper wire as disclosed in the above incorporated by reference patent applications, to include increase flexibility of the device. The electronics may have a much smaller footprint with custom ASIC that use the flex as a feedthrough, and we can use chip stacking to compress the electronic package to make the system flexible. Materials for electrode design, tissue ingrowth into the electrodes, etc. can also be used to anchor the system vs. hard anchors like sutures or bone screws. Moreover, communication can be done using BLE protocols along with the standard frequency shift key RF protocols, to allow more communication with the external power device. Such examples include smart phone cases, a case the plugs into a smart phone and that provides the RF transfer and logic via applications on the phone or an application controlled sticker that is attached to the cheek for quick use and controlled by the application on the phone.

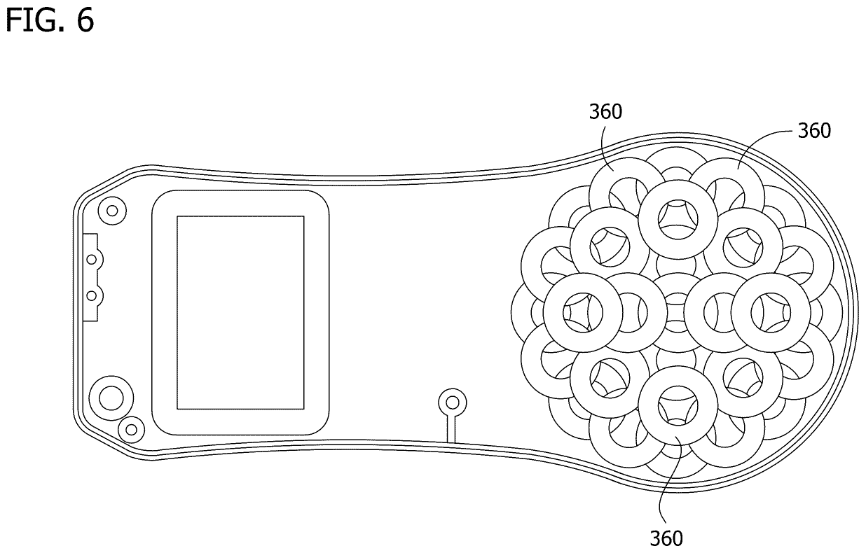

As shown in FIGS. 4 and 5, one embodiment might user a large coil 350 for powering the implant 310 allowing for the user to couple over a larger surface area. Referring to FIG. 6, another embodiment might use an array of smaller coils 360 arranged such that there is a large coupling area. Once the implant 310 has coupled to an individual coil, or multiple coils current to the unused coils could be turned off. By only powering the coupled coils, the efficiency of the system is improved, but more importantly there will be less thermal rise in patient applied part. These techniques for powering implants can be used without the documentation system as a standalone technology.

In addition to using RF for energy transfer, another embodiment of the implant could use ultrasound to power the implant. In this embodiment, the external remote would include a transducer and a small transducer in place of the RF coil in in the implant. A continuous or pulsed ultrasound signal could then be sent from the controller and the pressure wave could then be converted to electrical energy by the transducer in the implant. It is also considered that the communication between the implant and the remote could be modulated with the ultrasonic signal, or could be done through RF communications.

In addition, a capacitor or rechargeable power source could be integrated in the implant which would allow the implant to be charged and powered for standalone treatment for stroke patients who might be unable to hold the controller during treatment.

The energy consumption of the implant varies depending on the output of the neurostimulator and the operation of the device. To optimize power transfer, one embodiment of the implant could have additional capacitors to store energy with the current requirements of the implant are lower than the power received from the external controller. One embodiment could communicate with the controller to modulate the power being sent to the controller to match the consumption. Another embodiment could use a MOSFET or switch to disconnect the charging coil when the device is does not have active output and the onboard energy storage was sufficiently charged to power the ASIC. In another embodiment, the connection to the charge coil may have tri-state GPIO that can be used to uncouple the coil. When the reserved power dropped to a predetermined level or the power requirement of the system changes the coil would be switched back on so energy transfer from the handheld is restored. This will minimize the energy dissipation in the implant when powering the device without treatment. In another embodiment, the resonance frequency of the tuned coil can be altered by changing the capacitance of the circuit. This would lower the efficiency of the power transfer, but reduce the amount of energy required to be dissipated in heat when the output is not active.

In the case of vasospasm, in which the patients are hospitalized, the use of the therapy system may be automated for nurse/care giver control, not by the patient. In this case, the treatment may be applied several times per day for 15 minutes or longer while the patient is otherwise resting and may have suffered loss of function post stroke and post stroke intervention. The therapy system may be BLE controlled from a tablet and that can be periodically positioned near the patient to supply therapy without requiring the patient or car give to place something on the patient's body. In another example, a mat, a device positioned on the hospital bed, or otherwise positioned near the patient may be controlled from a nurse stand using BLE or other communication protocols that allow for long range control.

In addition to or in alternative to treating vasospasm, there are other areas that are involved stroke recovery. Neural stimulation to drive blood flow the brain, paired with AR/VR modalities that immerse the patient in therapeutic setting may cause the underlying brain matrix to change. Suitable AR/VR methods and systems are disclosed in U.S. Patent Application Publication No. 20190065970, the entirety of which is incorporated by reference herein. The matrix includes glia cells, neurons, etc. These cells need blood flow to remove the damaged from the stroke, or other diseases, and they need blood flow to cause healing and promote neural remodeling and plasticity. The stimulation would be timed to occur when the training environment is focused on activation of the specific neurological pathways that need to heal.

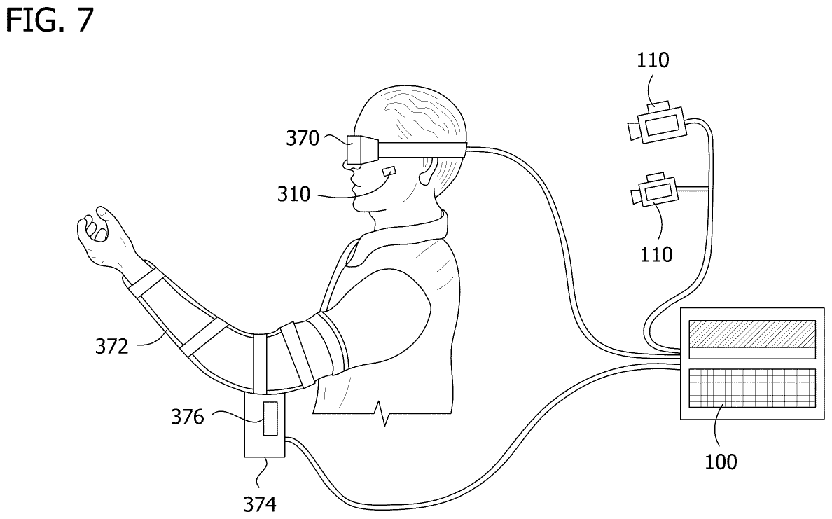

As shown in FIG. 7, for example, if the patient had a stroke, and lost function in their dominant hand, the device 310 may be implanted for initial intervention of the stroke and used for vasospasm treatment early. Then later treatment would be paired with AR/VR environment where the patient is focused on recovering hand/wrist motion, through immersive therapy in the AR/VR realm the patient will also receive stimulation to promote blood flow to the brain during the activity, hence leading increased recovery and increased outcomes. Such an example is shown in FIG. 7, wherein the patient wears VR goggles 370. A treatment device 372 (e.g., an orthosis or other range of motion device) may also be used, although it may not be used. The treatment device 372 may include a motor or other driver 374, although it may not include one. One or more sensors 376 may be associated with the driver 374, or the sensors may be independent of the motor whether the device includes a motor or does not include a motor. One embodiment of the system for using AR/VR in conjunction a neuromodulation implant may power the implant externally with the headset.

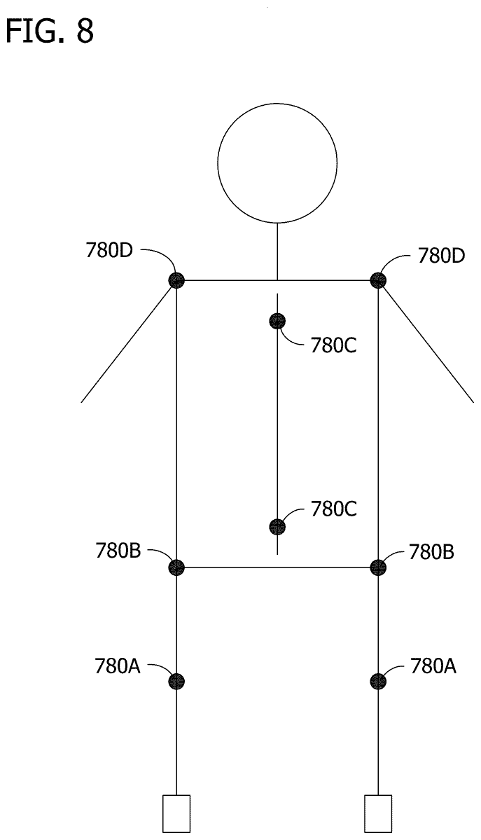

Other implanted sensors could be connected to the system as an input. The sensors may be powered externally via ultrasound, radiofrequency, or magnetic coupling. As shown in FIG. 8, examples of sensors 780A, 780B, 780C, 780D may be for knee, hip, spine, shoulder, respectively, or other musculoskeletal implants, which may be permanent or implanted for long term use. The wireless energy could be used for both powering the implant as well as data transfer using known encoding methods such as FSK and Manchester encoding. The power may be wirelessly sourced such as described above for the neurostimulation implant 310.

One Example of a Treatment for Use with System or Independent of System

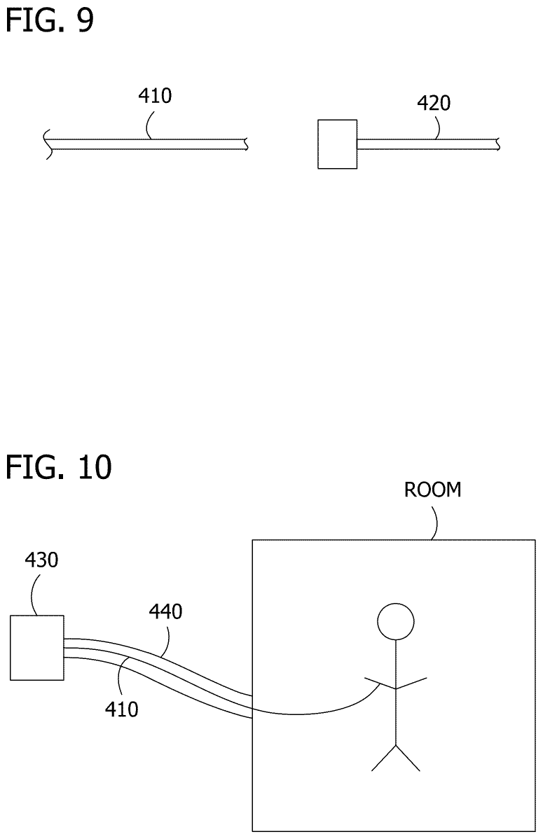

Referring to FIGS. 9 and 10, in one example, a suitable treatment for use with the documentation system or used independent of the system relates to an improved indwelling vascular access catheter 410 (i.e., a PICC or midline catheter) and use thereof. Currently a PICC or midline catheter, such as chemotherapy, requires a complex team and performed in surgery or radiology. A line is placed into a major vein through a cannula in the arm, and a guide wire is threaded through the line. The line is the removed and a triple lumen, indwelling vascular access catheter is threaded over the guide wire to the location near heart or into large central vein. The guidewire is then removed and often sutured in place. A whole team is required and it is expensive and time consuming. It is also very difficult to perform in an emergency. Further, the vascular access device is typically 18 gauge and the cannula in the arm is typically 14 gauge.

The improved vascular access catheter 410 is smaller than 18 gauge and can be delivered through a cannula 420 (e.g., a needle or peripheral IV line) that is smaller than 14 gauge. A cannula with a suitable design is disclosed in U.S. Pat. Nos. 9,168,163, and 9,498,249, the entirety of each of which is incorporated by reference herein. A vascular access catheter with a suitable design, although not a suitable gauge, is described in U.S. Patent Application Publication No. 2012/0296314, the entirety of which is incorporated by reference herein. The vascular access catheter 420 may be inserted into an arm (e.g., a vein such as cephalic, basilic, brachial, or median cubital veins in the upper arm) or other appendage of the patient and threaded so the distal tip is located in a central vein, or near or in the heart, or near or in the brain. Once the distal tip is properly positioned, medication can be delivered. Suitable medications can be anticoagulants like streptokinase required to dissolve a clot in the brain or in the heat, or a pulmonary embolism. This vascular access catheter 420 is used as a PICC or midline catheter to allow a rapid catheterization in an emergency and/or a cheaper and less efficient way of catheterization. A nurse or tech that can do an IV to be used as the cannula (e.g., cannula less than 14 gauge) and then thread the vascular access device from peripheral vein to near heart, for example. X-ray or fluoroscopy can confirm placement. It can be used in emergency treatment of non-hemorrhagic strokes or MI or PE as a midline or PICC (or other central) access catheter for rapid infusion of anticoagulants to dissolve clot and prevent further damage.

In one exemplary use, as shown in FIG. 10, the improved vascular access catheter 410 can extend outside a patient's room to an infusion system located outside the room. This will allow healthcare practitioners to operate the infusion system 430 (pump), e.g., add medications into the system, outside of the patient's room. The vascular access catheter 410 can be run under a door or through a small passage in wall. A protection sleeve 440 can be placed around the vascular access catheter 410 at locations where the catheter is under the door or through a passage or on floor so if stepped on or pressure doesn't kink line at those critical areas. Thus fluid flow from infusion pump 430 through the vascular access catheter 410 maintains pressure and will not be kinked or bent with protective sleeve. The infusion pump 430 is disposed outside room for safety of staff who add complex and expensive medications safely. Also, because the vascular access catheter 410 has a small lumen, in one embodiment only 5 cc or less may be necessary to flush the vascular access catheter.

Other Audio and/or Visual Embodiments

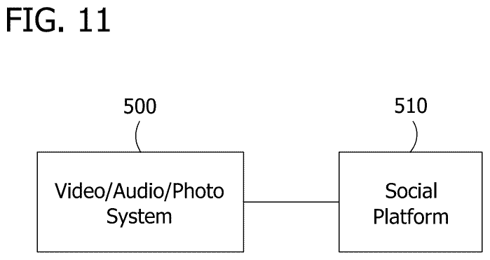

In another embodiment, shown in FIG. 11, an audio and/or visual editing and sharing application or platform 510 allows connected users to share video, audio, and/or image, edit the shared video, audio, and/or images from their system 500, and share the edited shared video, audio, and/or images. Thus, multiple people can add to creativity in short pieces or segments. Editing tools allow insertion at segments to augment add or subtract to a stream to improve or change a “creation.” Users can vote or comment. This brings a large group of people into a collaboration. For example, a picture, a word, a video segment, song, and/or a note/rhythm/beat can be added to see if you can make something more popular in combinations then share with other users to see if better or more popular. This can be incorporated into an application like TikTok or YouTube. Each individual's contribution to the media could be weighted by the impact it has on the total amount of likes or shares that a video has. In one embodiment this could be tracked by the time the person has spent editing the video, by the timing of the responses that (likes, ratings, etc.) based on the individual's contribution, by the increase of responses after a contribution, or any combination of these or other metrics. This would allow the distribution of revenue from advertisement to be done proportionally in exchange for releasing the creator's rights under DMCA. In addition, there could be a ranking of contributors based on popularity of the popularity of the media that they created. The software could allow for the video editing could be controlled via traditional input or voice control.

In another embodiment a physician's mouse patterns will be captured during normal using software. These movements will be compiled over time and then used to predict the user's patterns of using software such as electronic medical records. After enough data has been compiled to predict the usage patterns of the user the software can update the mouse position to the predicted field or position that the user would need next. This could be useful to maximum physician productivity. This could be used with other applications including, but not limited to gaming, office applications, surgical planning software, web browsers, and phone apps.

Modifications and variations of the disclosed embodiments are possible without departing from the scope of the invention defined in the appended claims.

Embodiments of the present disclosure may comprise a special purpose computer including a variety of computer hardware, as described in greater detail below.

For purposes of illustration, programs and other executable program components may be shown as discrete blocks. It is recognized, however, that such programs and components reside at various times in different storage components of a computing device, and are executed by a data processor(s) of the device.

Although described in connection with an exemplary computing system environment, embodiments of the aspects of the invention are operational with other special purpose computing system environments or configurations. The computing system environment is not intended to suggest any limitation as to the scope of use or functionality of any aspect of the invention. Moreover, the computing system environment should not be interpreted as having any dependency or requirement relating to any one or combination of components illustrated in the exemplary operating environment. Examples of computing systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Embodiments of the aspects of the invention may be described in the general context of data and/or processor-executable instructions, such as program modules, stored one or more tangible, non-transitory storage media and executed by one or more processors or other devices. Generally, program modules include, but are not limited to, routines, programs, objects, components, and data structures that perform particular tasks or implement particular abstract data types. Aspects of the invention may also be practiced in distributed computing environments where tasks are performed by remote processing devices that are linked through a communications network. In a distributed computing environment, program modules may be located in both local and remote storage media including memory storage devices.

In operation, processors, computers and/or servers may execute the processor-executable instructions (e.g., software, firmware, and/or hardware) such as those illustrated herein to implement aspects of the invention.

Embodiments of the aspects of the invention may be implemented with processor-executable instructions. The processor-executable instructions may be organized into one or more processor-executable components or modules on a tangible processor readable storage medium. Aspects of the invention may be implemented with any number and organization of such components or modules. For example, aspects of the invention are not limited to the specific processor-executable instructions or the specific components or modules illustrated in the figures and described herein. Other embodiments of the aspects of the invention may include different processor-executable instructions or components having more or less functionality than illustrated and described herein.

The order of execution or performance of the operations in embodiments of the aspects of the invention illustrated and described herein is not essential, unless otherwise specified. That is, the operations may be performed in any order, unless otherwise specified, and embodiments of the aspects of the invention may include additional or fewer operations than those disclosed herein. For example, it is contemplated that executing or performing a particular operation before, contemporaneously with, or after another operation is within the scope of aspects of the invention.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively or in addition, a component may be implemented by several components.

The above description illustrates the aspects of the invention by way of example and not by way of limitation. This description enables one skilled in the art to make and use the aspects of the invention, and describes several embodiments, adaptations, variations, alternatives and uses of the aspects of the invention, including what is presently believed to be the best mode of carrying out the aspects of the invention. Additionally, it is to be understood that the aspects of the invention is not limited in its application to the details of construction and the arrangement of components set forth in the following description or illustrated in the drawings. The aspects of the invention are capable of other embodiments and of being practiced or carried out in various ways. Also, it will be understood that the phraseology and terminology used herein is for the purpose of description and should not be regarded as limiting.

As various changes could be made in the above constructions, products, and methods without departing from the scope of the invention, it is intended that all matter contained in the above description and shown in the accompanying drawings shall be interpreted as illustrative and not in a limiting sense. In the preceding specification, various preferred embodiments have been described with reference to the accompanying drawings. It will, however, be evident that various modifications and changes may be made thereto, and additional embodiments may be implemented, without departing from the broader scope of the aspects of the invention as set forth in the claims that follow. The specification and drawings are accordingly to be regarded in an illustrative rather than restrictive sense.

In view of the above, it will be seen that several advantages of the aspects of the invention are achieved and other advantageous results attained.

The Abstract and Summary are provided to help the reader quickly ascertain the nature of the technical disclosure. They are submitted with the understanding that they will not be used to interpret or limit the scope or meaning of the claims. The Summary is provided to introduce a selection of concepts in simplified form that are further described in the Detailed Description. The Summary is not intended to identify key features or essential features of the claimed subject matter, nor is it intended to be used as an aid in determining the claimed subject matter.

What is claimed is:

1. A video documentation system comprising:
at least one camera configured to capture video of an event and to generate event data representative thereof;
one or more processors coupled to the camera, the one or more processors receiving and responsive to the event data via a communications network; and
one or more non-transitory computer-readable media coupled to the one or more processors, the one or more non-transitory computer-readable media storing:
an artificial intelligence (AI) system configured to generate a record of one or more critical activities occurring during the event; and
instructions that, when executed by the one or more processors, configure the system to perform operations, the operations comprising:
receiving, by the AI system, the event data representative of the event from the at least one camera;
processing the received event data with the AI system to identify the one or more critical activities; and
providing the record of the one or more critical activities occurring during the event as an output of the AI system.

2. The system of claim 1, wherein processing the received event data with the AI system to identify the one or more critical activity comprises differentiating at least one non-critical activity from the one or more critical activities.

3. The system of claim 1, wherein the at least one camera and the one or more processors are in wireless communication with one another via the communications network.

4. The system of claim 1, wherein the AI system implements one or more of predictive learning, machine learning, automated planning and scheduling, machine perception, computer vision and affective computing to generate the record.

5. The system of claim 1, wherein the event data comprises both video data and audio data associated with the video data, and wherein the instructions, when executed by the one or more processors, further configure the system to perform operations, the operations comprising:
receiving, by the AI system, the audio data representative of the event; and
processing the received audio data with the AI system to recognize spoken words.

6. The system of claim 5, wherein the one or more critical activities includes a timeout activity initiating a medical procedure event, and wherein the spoken words recognized by the AI system include at least one of the following: a patient's name; a medical procedure to be performed; a time of day; and a date.

7. The system of claim 6, wherein the instructions, when executed by the one or more processors, further configure the system to perform operations, the operations comprising:
processing, by the AI system, the words recognized by the AI system to verify the timeout activity before continuing the medical procedure event.

8. The system of claim 1, wherein the non-transitory computer-readable media comprises a database containing information relating to a plurality of past events, and wherein the instructions, when executed by the one or more processors, further configure the system to perform operations, the operations comprising:
accessing, by the AI system, the information in the database;
processing the information in the database with the AI system to identify a template for the event; and
processing the received event data with the AI system to determine if the one or more critical activities identified in the event data substantially conform to the template.

9. The system of claim 1, wherein the one or more critical activities includes visualization of a target tissue, wherein the non-transitory computer-readable media comprises a database containing information relating to a plurality of past events, and wherein the instructions, when executed by the one or more processors, further configure the system to perform operations, the operations comprising:
accessing, by the AI system, the information in the database;
processing the information in the database to identify at least one of a pre-operative, intra-operative, and post-operative analysis of the event; and
processing the received event data with the AI system to determine if the visualization of a target tissue substantially conforms to the at least one of the pre-operative, intra-operative, and post-operative analysis.

10. The system of claim 1, further comprising a device for applying electrical stimuli to a patient, wherein the one or more critical activities include application of the electrical stimuli, and wherein the instructions, when executed by the one or more processors, further configure the system to perform operations, the operations comprising:
receiving, by the AI system, the event data representative of the event; and
processing the received event data with the AI system to identify the application of the electrical stimuli and the patient's response thereto.

11. A method of generating a record of critical activities occurring during an event, the method comprising:
receiving, by an artificial intelligence (AI) system, event data representative of the event, wherein the event data is received from at least one camera configured to capture video of the event and to generate the event data representative thereof; and
executing, by one or more processors, instructions stored on one or more non-transitory computer-readable media to configure the AI system to perform operations, the operations comprising:
processing the received event data with the AI system to identify the critical activities; and
providing the record of the one or more critical activities occurring during the event as an output of the AI system.

12. The method of claim 11, wherein processing the received event data with the AI system to identify the one or more critical activity comprises differentiating at least one non-critical activity from the one or more critical activities.

13. The method of claim 11, wherein the at least one camera is coupled to the one or more processors via a wireless communications network.

14. The method of claim 11, wherein the AI system implements one or more of predictive learning, machine learning, automated planning and scheduling, machine perception, computer vision and affective computing to generate the record.

15. The method of claim 11, wherein the event data comprises both video data and audio data associated with the video data, and further comprising:
receiving, by the AI system, the audio data representative of the event; and
processing the received audio data with the AI system to recognize spoken words.

16. The method of claim 15, wherein the one or more critical activities includes a timeout activity initiating a medical procedure event, and wherein the spoken words recognized by the AI system include at least one of the following: a patient's name; a medical procedure to be performed; a time of day; and a date.

17. The method of claim 16, further comprising processing, by the AI system, the words recognized by the AI system to verify the timeout activity before continuing the medical procedure event.

18. The method of claim 11, further comprising:
accessing, by the AI system, the information in a database containing information relating to a plurality of past events;
processing the information in the database with the AI system to identify a template for the event; and
processing the received event data with the AI system to determine if the one or more critical activities identified in the event data substantially conform to the template.

19. The method of claim 11, wherein the one or more critical activities includes visualization of a target tissue, and further comprising:
accessing, by the AI system, the information in a database containing information relating to a plurality of past events;
processing the information in the database with the AI system to identify at least one of a pre-operative, intra-operative, and post-operative analysis of the event; and
processing the received event data with the AI system to determine if the visualization of a target tissue substantially conforms to the at least one of the pre-operative, intra-operative, and post-operative analysis.

20. The method of claim 11, wherein the one or more critical activities include application of electrical stimuli to a patient, and further comprising:
receiving, by the AI system, the event data representative of the event; and
processing the received event data with the AI system to identify the application of the electrical stimuli and the patient's response thereto.
